(19) United States
(12) Patent Application Publication
(10) Pub. No.: US 2025/0378909 A1
(43) Pub. Date: Dec. 11, 2025

(54) NON-INVASIVE METHOD FOR DETERMINING PRENATAL PARENTAGE RELATIONSHIPS USING MICROHAPLOTYPES

(71) Applicant: XIAMEN VANGENES BIOTECHNOLOGY CO., LTD., Fujian (CN)

(72) Inventors: Hongliang CHEN, Fujian (CN); Yihan LI, Fujian (CN); Hailing ZHENG, Fujian (CN); Xingqiang ZHU, Fujian (CN); Huan XU, Fujian (CN); Yue XIAO, Fujian (CN); Ping GUO, Fujian (CN); Zhi HE, Fujian (CN); Shuling WU, Fujian (CN); Mengting WU, Fujian (CN)

(73) Assignee: XIAMEN VANGENES BIOTECHNOLOGY CO., LTD., Fujian (CN)

(21) Appl. No.: 18/878,607

(22) PCT Filed: Jul. 17, 2023
(86) PCT No.: PCT/CN2023/107665

§ 371 Date: Dec. 23, 2024

(30) Foreign Application Priority Data
Jun. 24, 2022 (CN) 202210728875.8

(51) Int. Cl. G16B 20/20; G16B 20/50; G16B 25/20; G16B 35/20

(52) U.S. Cl. CPC G16B 20/20; G16B 20/50; G16B 25/20; G16B 35/20

(57) ABSTRACT

The present invention provides a non-invasive method for determining prenatal parentage relationship using microhaplotypes. Specifically, the present invention utilizes a method for screening sites, which includes pre-filtrating, identifying microhaplotypes, statistically analyzing genetic parameters of microhaplotype populations, and Hardy-Weinberg equilibrium testing.

### TECHNICAL FIELD

The present invention relates to the field of genetics technology. Specifically, the present invention relates to a non-invasive method for determining prenatal parentage relationships using microhaplotypes. More specifically, the present invention employs a novel method for screening sites.

### BACKGROUND ART

In 2012, Professor Kidd's research team at Yale University in the United States selected SNPs with relatively close positions from regions less than 10 KB in length based on previous haplotype related research, and avoided recombination prone sites, ultimately screening out 8 mini-haplotype loci. Through testing 45 populations, the results showed that the high heterozygosity and population distribution differences of the selected 8 mini-haplotype loci can provide relevant information for parentage identification and racial inference. In order to further screen for haplotype loci that are more suitable for forensic applications. In 2013, Professor Kidd's research team at Yale University selected sequence fragments within 300 bp and containing at least 2 SNP sites from existing genome databases, and named them as microhaplotypes (MH). Microhaplotypes combine the advantages of STR (Short Tandem Pepeats) and SNP (Single Nucleic Polymorphism):
\textcircled{1} high polymorphism: typically. SNP sites have only 2 alleles, and microhaplotypes composed of multiple SNPs theoretically have higher complexity;
\textcircled{2} low mutation rate: the mutation rate of microhaplotypes is equivalent to that of SNPs, is 10⁻⁸/generation, which is one millionth to one hundred thousandth of the mutation rate of STR, having a unique advantage in parentage identification;
\textcircled{3} detection without shadow bands: STR based on electrophoresis technology typing will produce shadow bands, which is not conducive to the analysis of complex mixed DNA samples. Microhaplotypes are detected through sequencing methods without shadow bands, and second-generation sequencing has the advantages of high throughput and high sensitivity, which has great potential in quantitative analysis of complex mixed DNA;
\textcircled{4} length advantage: STR loci have a large range of allele lengths, which can lead to amplification imbalance. Longer alleles are highly likely to be disrupted in degraded samples, resulting in inaccurate typing results. The length of microhaplotypes is relatively uniform, which can reduce amplification imbalance caused by length differences.

Prenatal fetal parentage identification includes invasive sampling based on chorionic or amniocentesis, which may cause infection or even miscarriage, and the puncture time is limited; currently, non-invasive prenatal parentage identification based on peripheral blood sampling has gradually become the primary choice.

In 1997, Professor Yuming Lu discovered the presence of fetal free DNA in the peripheral blood plasma of pregnant women. With the development of high-throughput sequencing, non-invasive prenatal fetal parentage identification using SNPs as genetic markers appeared in the market after 2013. As described in patent CN104946773A, 1035 SNPs were successfully used for prenatal fetal genetic diagnosis, but due to limitation to SNPs as a binary genetic marker, a large quantity is required. With the discovery of microhaplotypes, which have the advantages of SNPs, simultaneously also have high polymorphism, they are naturally considered as genetic markers for prenatal fetal parentage identification, for example, CN111518917A utilizes 60 microhaplotypes as markers for prenatal parentage identification. This patent verifies the feasibility of microhaplotypes in prenatal scene, but only makes preliminary attempts. This patent further expands the scope of the patent sites and makes significant innovations in data processing, identification methods, and application scenarios.

### SUMMARY OF THE INVENTION

The purpose of the present invention is to provide a method for parentage identification using peripheral blood of pregnant women during pregnancy, which makes further innovation and deepening on the basis of the preliminary attempts made in the existing technology.

In one aspect, the present invention provides a method for screening sites, characterized in that the method comprises the steps of:
(1) pre-filtrating:
(2) identifying microhaplotypes;
(3) statistically analyzing genetic parameters of microhaplotype populations:
(4) Hardy-Weinberg equilibrium testing.

Preferably, in step (1): the VCF files of a certain population or all races in the Thousand Genomes Project contain all mutation data, in allele frequency of the selected population, the minor allele frequency is greater than 0.01; SNPs are located no autosomes and include tiny insertion and deletion.

Preferably, in step (1), insertion and deletion (inDel) can be filtered out according to a sequencing platform.

Preferably, in step (2), all pre-filtered SNPs are sorted by position, and the first SNP is defined as “start SNP” and combined sequentially with the subsequent SNPs, if a gap with the “start SNP” is within 350 bp, then they are combined to form a microhaplotype, with “start SNP” and the number of SNPs being as unique markers;
if the gap between the SNP and the “start SNP” exceeds 350 bp, marking the SNP next to the original “start SNP” as the “start SNP” and performing the above combination to identify each SNP in sequence;
for the microhaplotype of a certain “start SNP” that may combine more than 2 SNPs, selecting the one with the most SNPs as the complete set and the others as subset, then removing the subset;
if the gap the between “start SNP” of the adjacent microhaplotypes may be less than 350, that is, microhaplotypes partially overlap, and should be retained for now.

Preferably, in step (2), 350 bp can be adjusted according to the selected reagents and experimental conditions. For example, in prenatal parentage identification, due to the short cf-DNA fragment, it is more suitable to select 70-150 bp.

Preferably, in step (3), for the identified microhaplotypes described above, the information of each SNP can be found in the VCF file of (1), the effective number of alleles (Ae), informativeness (In), and allele frequency (P) of each microhaplotype are counted, if the Ae value of a certain genetic marker is n, it means that the genetic marker is equivalent to containing n alleles that have equal frequencies, that is, the frequency of each allele is 1/n.

Preferably, the calculation formula for Ae value is 1/Σpi², where pi represents the frequency of allele i on a certain locus, for the overlapping microhaplotypes in (2), the one with higher Ae/Nₛₙₚ value are retained.

Preferably, in step (4), for the selected microhaplotypes, Pearson chi square test is used to perform Hardy-Weinberg equilibrium test on the genotype distribution frequency of the microhaplotypes, and non-matching microhaplotype combinations are marked for selection based on subsequent applications.

Preferably, after this step is completed, there are several millions of microhaplotypes, selecting is performed based on length, Ae, chromosome, and identification requirements.

Preferably, in step (4), based on other research experience, two microhaplotypes with gap of over 10 kb are selected.

In another aspect of invention, the present invention provides a non-invasive method for determining prenatal parentage relationship using microhaplotypes, characterized in that the non-invasive method for determining prenatal parentage relationships comprises any one of the methods for screening sites as described above.

Preferably, the method comprises calibrating the background noise of sequencing, wherein calculating the background error by the following: the alignment results were call snp by genotyping software such as GATK to obtain a vcf file, after removing SNPs contained in all the microhaplotypes, the number of bases in the remaining SNPs that were inconsistent with the reference genome was counted, and divided by the total number of bases aligned to the microhaplotypes in the sample. Statistical and calibration methods for background errors can also be achieved by methods such as adding UMI.

Preferably, the method comprises calculating fetal concentration: adding probes covering the Y chromosome and using the proportion of the Y chromosome to calculate fetal concentration, denoted as FF_{y}; using the software FetalQuant to calculate fetal concentration; using SeqFF algorithm to calculate fetal concentration; using cfDNA fragment length information to calculate fetal concentration; using the Nucleosome track method to calculate fetal concentration; using methylation to calculate fetal proportion and the like.

Preferably, the method comprises an analysis method for sample contamination: evaluating whether the sample is contaminated by genotypes that are not at a reasonable frequency in male samples, genotypes can be marked by microhaplotypes or can be marked by SNPs to analyze whether the sample is contaminated.

Preferably, the method comprises an identification method, which uses t-test, P-value to determine genetic relationship.

Preferably, the non-invasive method for determining prenatal parentage relationship is used to analyze whether the fetus in singleton, twin, dizygotic twin, and assisted reproduction has mistaken sperm and/or egg and so on.

### Notable Progress of the Present Patent

The present invention is a method for parentage identification using peripheral blood of in pregnant women during pregnancy, compared with the puncture sampling method, it has the advantages of non-invasive identification process and convenient sampling and mailing; compared with existing methods that utilize SNPs, the method of the applicant reduces the need for sites and thus lowers costs due to the use of microhaplotypes as markers; and microhaplotypes have the advantage of having multiple alleles, and have the ability to identify complex mixed samples such as dizygotic twins, which compensates for the shortcomings of SNPs.

The present invention provides a specific and feasible solution for the identification process, establishes a comprehensive quality control, and offers solutions to various problems that may arise in reality.

### DETAILED DESCRIPTION

The following provides a detailed description of the technical solution of the present invention in combination with Examples and tables, but does not limit the present invention to the scope of the Examples as described.

The present invention has been innovated in the following four aspects:

1. Microhaplotype sites: the present invention utilizes a novel method for screening sites; at present, microhaplotypes are linear combinations of 2 or more SNP sites, which have been expanded to include snp+snp, snp+str, snp+inDel. The specific screening is as follows:

(1) Pre-filtrating: The VCF files of a certain population (such as the Han ethnic group in southern China) or all races in the Thousand Genomes Project contain all mutation data. In this Example, the Han ethnic group in southern China is selected, and the population with the minor allele frequency (MAF) greater than 0.01 is selected; SNPs are located on autosomes and include tiny insertions and deletions.

(2) Identifying microhaplotypes: all pre-filtered SNPs are sorted by position, and the first SNP is defined as “start SNP” and combined sequentially with the subsequent SNPs, if the gap with the “start SNP” is within 350 bp, then they are combined to form a microhaplotype, with “start SNP” and the number of SNPs being as unique markers; if the gap between the SNP and the “start SNP” exceeds 350 bp, marking the SNP next to the original “start SNP” as the “start SNP” and performing the above combination to identify each SNP in sequence; for the microhaplotype of a “start SNP” that may combine more than 2 SNPs, selecting the one with the most SNPs as the complete set and the others as subsets, then removing the subsets; if the gap the between “start SNP” of the adjacent microhaplotypes may be less than 350, that is, microhaplotypes partially overlap, and should be retained.

(3) Statistically analyzing genetic parameters of microhaplotype populations: for the identified microhaplotypes described above, the information of each SNP can be found in the VCF file of (1), counting the effective number of alleles (Ae), informativeness (In), and allele frequency (P) of each microhaplotype. The effective number of alleles (Ae) is a classic concept in population genetics, value of which represents the number of alleles of equal frequency that are equivalent to a genetic marker.

For example, if the Ae value of a certain genetic marker is n, it means that the genetic marker is equivalent to containing n alleles with equal frequencies, that is, the frequency of each allele is 1/n. Comparison and ranking for genetic markers of multiple alleles can be achieved by this marker. The calculation formula for Ae value is 1/Σpi², where pi represents the frequency of allele i on a certain locus, for the overlapping microhaplotypes in (2), the one with higher Ae/Nsnp value are retained.

(4) Hardy-Weinberg equilibrium test: for the selected microhaplotypes, Pearson chi square test is used to perform Hardy-Weinberg equilibrium test on the genotype distribution frequency of the microhaplotypes, Hardy-Weinberg equilibrium refers to the absence of significant differences between observed value and theoretical value of genotype distribution frequencies (P>0.05). Non-matching microhaplotype combinations are marked for selection based on subsequent applications. After this step is completed, there are several millions of microhaplotypes, and selection was performed based on length, Ae, chromosome, and identification requirements.

Alternatively, (1) insertion and deletion of inDel can be filtered out based on the sequencing platform.

Alternatively, (2) 350 bp can be adjusted according to the selected reagents and experimental conditions. For example, in prenatal parentage identification, due to the shorter cf-DNA fragment, it is more suitable to select within 70-150 bp.

Alternatively, in (4), based on other research experience, two microhaplotypes with gap of over 10 kb are selected.

2. Data processing: normal analysis methods, the applicant has also developed calibration for sequencing background noise, as different platforms have their own characteristics and require targeted calibration; calculation of fetal concentration: in prenatal parentage identification, estimation of fetal concentration is crucial for fetal genotyping. Fetal concentration is an important quality control, so the applicant has developed a set of quantitative method for fetal concentration; sample contamination: in prenatal parentage identification, samples such as nails and hair in male samples are prone to be contaminated during collection and transportation, and may even be contaminated during the experimental stage. Therefore, the applicant has also developed a set of analysis method for sample contamination. Other methods require testing of pregnant women's white blood cells to identify their genotypes, while the applicant can obtain maternal and child typing by combining fetal concentration with cfDNA from pregnant women, and only two samples are used on the machine, which significantly reduces costs. The above methods are detailed recorded in the description.

3. Identification method: the calculation method of CPI is similar to the method with traditional STR as a marker, which is known to appraisers as a method to identify forensic physical evidence; in addition to using this method, the applicant has also developed a set of methods that utilize t-test and P-value to determine parentage relationship. This method can calculate more quickly and is more convenient for cases with a large number of microhaplotypes, without considering specific frequencies and rare genotypes.

4. In addition to common singleton, the applicant also analyzes whether the fetus has mistaken sperm and/or egg and so on in twin, dizygotic twin, and assisted reproduction. With the increasing proportion of infertility, the population of assisted reproduction is also growing. In this case, the proportion of dizygotic twins increases, and the demand for whether the egg donor or sperm donor has parentage relationship with the fetus will also increase.

### Embodiments

1. Screening sites: this Example is based on the ion proton platform, taking into account the characteristics of the proton platform. Based on the selected microhaplotypes (see the previous content for specific steps), selecting a total of 348 loci with a length less than 160 bp and absence of continuous repeat bases near SNPs in the internal sequences of microhaplotypes.

2. Probe synthesis: organizing the position information of each microhaplotype into a bed file format and submitted to NAANGDA (Nanjing) Biotechnology Co., Ltd. for design and synthesis by NAANGDA.

3. Nucleic acid extraction: performing nucleic acid extraction first after receiving the sample to be identified.

4. End Repair: mixing the mixed DNA fragments obtained in step 1, End Repair Buffer, and End Prep Enzyme, and placing it in a PCR instrument after vortexing for reaction at the following temperatures: incubating at 20° C. for 15 minutes and incubating at 65° C. for 15 minutes.

5. Adapter ligation: directly adding Rapid Ligation Buffer 2, Ligation Enzyme Mix 2, and adapters to the product of end repair in step 2, and placing them in a PCR instrument after vortexing for reaction at the following temperatures: incubating at 22° C. for 30 minutes, incubating at 68° C. for 5 minutes, and incubating at 72° C. for 5 minutes.

6. Library purification: purifying the product obtained in step 3 to obtain DNA fragments with adapters added.

7. PCR amplification: mixing the mixed DNA fragments obtained in step 4, PCR Primer Mix, and Amplification Mix 3 and performing PCR amplification and purification to obtain the desired target library.

8. Library detection: detecting the amplification product obtained in step 5 for library concentration and fragment size using qubit and Agilent 2100.

9. Preparation before hybridization: mixing all libraries obtained in step 6 by equal mass, to which adding blocker and Cot-1 human DNA, and concentrating them into dry powder in a concentrator at 70° C.

10. Hybridization capture: adding 2×Hybridization buffer (vial 5) and Hybridization component A (vial 6) into the dry powder tube in step 7, and incubating at room temperature for 5 minutes followed by addition of the probe designed in step 2. After vortexing and mixing well, placing it in the PCR instrument for hybridization at the following temperature: hybridization at 65° C. for 4-16 hours.

11. Hybridization elution: eluting the hybridized mix sample after hybridization to obtain the target sequence.

12. High throughput sequencing: performing high-throughput sequencing on the target sequence obtained in the previous step.

13. Data preprocessing: using software fastp to perform quality filtering, removing low-quality sequencing sequences and removing low-quality sequences; other quality filtering software is also acceptable.

14. Sequence alignment: using the sequence alignment software BWA (Burrows Wheeler Aligner Multi vision Software Package) to align the sequence obtained from the above steps with the human reference genome (hg19 version) sequence; in the previous step, other alignment software such as soap, bowtie2, etc., can be selected, and other versions of reference genome versions can be selected and used.

15. Sample microhaplotyping: when a sequence is aligned into the genomic range/interval of a certain microhaploid, it is considered as the target sequence of the microhaploid. In the SAM format alignment file of the sequence, the base types of all SNPs of the sequence in the microhaploid are extracted by a script written in Python and combined to obtain the typing of the sequence in the microhaploid;
alternatively, due to the low sequencing quality of proton platform, each sequence that is aligned into the microhaplotype is further filtered to remove the sequences of the 3 bases of SNP at the beginning and end of the sequences; the sequences containing insertions and deletions within the 3 bp base range before and after the target SNP are removed. 3 it is adjustable according to the actual situation.

16. Statistics of microhaploid typing and genotype frequency: counting all typing type alles, and corresponding alle numbers (AN) and alle frequencies (AF) of each microhaploid, wherein the frequency is the number of allelic genotyping AN/the number of all typing of the microhaploid.

17. Analysis background errors: due to the possible presence of replication errors during the PCR replication process, when sequencing is carried out, sequencing errors may cause background errors of an analysis. Analyzing background errors can help calculate fetal concentrations and also provide quality control for sequencing data.

Background error calculation: the alignment results were call snp by genotyping software such as GATK to obtain a vcf file, after removing SNPs contained in all the microhaplotypes, the number of bases in the remaining SNPs that were inconsistent with the reference genome was counted, and divided by the total number of bases aligned into the microhaplotypes in the sample. Counting background errors are common quality control steps in NGS data analysis. This step only lists one of the methods and other algorithms or software can also achieve this goal; more preferably, counting and calibration of background errors can be achieved by methods such as adding UMI.

18. Whether male sample is contaminated: if a male sample is not contaminated, each microhaplotype is generally homozygous or heterozygous. Considering the chain preference during PCR, the frequency of a certain genotype in males at the heterozygous sites will not be lower than 0.2; considering background errors in homozygous sites, the dominant genotype frequency is generally not lower than 95%. Therefore, numbers of genotypes with AF_{heterozygosity} between 0.05-0.2 and the numbers of AF_{homozygosity} greater than 95% are all counted, the contamination index is AF_{heterozygosity}/AF_{homozygosity}. The contamination index for uncontaminated males will be lower than 10%. If it is higher, the sample will be considered as contaminated, and the contamination proportion can be quantified based on the numerical value. The specific ratio of this step can be determined based on the actual platform and number of layers. The main idea is to evaluate whether a sample is contaminated by genotypes that are not at a reasonable frequency in male samples. Genotypes can be marked by microhaplotypes or SNPs to analyze whether the sample is contaminated.

19. Calculating fetal concentration: the SNP or microhaplotype frequency in cfDNA carries fetal concentration information. Referring to CN104846089A, the fetal concentration is calculated, denoted as FFₛₙₚ.

Step 19 alternatively, probes that cover the Y chromosome are added to calculate fetal concentration using the proportion of the Y chromosome, denoted as FF_{y}; software FetalQuant is used to calculate fetal concentration; SeqFF algorithm is used to calculate fetal concentration; cfDNA fragment length information is used to calculate fetal concentration; nucleosome track method is used to calculate fetal concentration; and methylation is used to calculate fetal proportion and other methods.

20. Male microhaplotype typing: typically, in male genomic DNA, if one of genotype frequencies is greater than 0.9, the microhaplotype is considered as homozygous; if the genotype frequency ratio is between 0.2-0.8, it is considered as heterozygous. If the contamination index is higher in step 20, the possible genotype can be calculated based on the contamination proportion.

21. Microhaplotype typing of pregnant women: (accurate typing is a prerequisite for subsequent analysis of parentage relationships) pregnant women and fetuses can be typed according to the genotype frequency by analyzing the data of free DNA in the peripheral blood of pregnant women, and counting the frequency of each genotype of microhaplotypes according to step 16 followed by filtering out genotypes that may be caused by background errors. If a certain microhaplotype has more than one genotype: the gene frequency is less than half of the maximum gene frequency of the microhaplotype and greater than the genotype with background errors. In combination with the fetal concentration calculated earlier, whether to retain the genotype is determined, and this site is recorded. The number of such microhaplotypes in each sample is counted and recorded as sample M_{two}.

The specific typing method in the above step: for each microhaplotype of free DNA in pregnant women, there are four possible combinations of mother and child themselves: genotype set K: {PPpp, PPpq, PQqq, PQpq}, uppercase letters represent mother, lowercase letters represent fetus, P represents a certain genotype, and Q represents all genotypes except P. For each microhaplotype, the frequency P_{f} of each combination of maternal and fetal genotypes is calculated according to the fetal concentration calculated in step 14. According to the actual allele frequency Pₜ calculated in step 11, each microhaplotype maternal and fetal typing is obtained through the maximum likelihood method.

Preferably, for the method of mother and fetus typing: without considering the fetal concentration calculated in step 19, the fetal concentration range is set at an interval of 0.5% (1-20%) based on experience, and the fetal concentration is set to 1% in sequence. Given the combination K of mother and fetus in each microhaplotype, the frequency P_{fk(1:4)} of each combination of mother and fetus genotype is calculated based on the set fetal concentration, k is one of the sets K, and the microhaplotype is typed into one of seven P_{fk} based on the actual allele frequency. The typing of the microhaplotype is recorded, and a difference between theoretical and actual frequencies Error=|P_{fk}−Pₜ| is calculated. The sum ΣError of the frequency differences of all microhaplotypes is counted. Then the fetal concentration is increased 0.5% in order, the sum ΣError of frequency differences for all concentrations is calculated, and the fetal concentration corresponding to the minimum sum of frequency differences is selected. The typing of all microhaplotypes at this concentration is the final typing.

The preferred typing method in step 21: allele reads data of each microhaplotype serves as input and the maternal and fetal genotypes of a single microhaplotype are predicted based on Bayesian algorithm. The model obtains the maximum expectation through exhaustively listing fetal concentration iteration. Specifically, a probability of belonging to each genotype is first simulated based on reads value of a certain input: p(aᵢ|Gᵢ=k,Ni,μ₁:7)˜Binom(aᵢ|μₖ,Nᵢ). For each microhaplotype i. Gi represents the genotype i therein, Nᵢ represents the number of all sequences aligned to this haplotype, aᵢ represents the number of sequences supported by a certain genotype, and μₖ is a given parameter. Given θ=(μ₁:, π), according to Bayesian algorithm, the calculated probability is used to calculate the posterior probability

$${y}_{i} ( k ) : {y}_{i} ( k ) = p ( G = k \vert {a}_{i} , {N}_{i} , θ ) == \frac{{π}_{k} Binom ( {a}_{i} \vert {μ}_{k} , {N}_{i} )}{{\sum }_{j = 1}^{K} {π}_{j} Binom ( {a}_{i} \vert {μ}_{j} , {N}_{i} )} ,$$

among them, πₓ refers to the genotype frequency of genotype k, which has been preserved during screening sites. This method refers to SNVMix2 (Goya R, Sun M G, Morin R D, et al. SNVMix: predicting single nucleotide variants from next-generation sequencing of tumors. Bioinformatics. 2010; 26 (6): 730-736), wherein the specific parameters that have been modified are as follows:

| | | Expected reference | | |
|---|---|---|---|---|
| Genotype^{a} | δ | allele frequency | αₖ | βₖ |
| PPpp | p³ | 1 | 1000 | 1 |
| PPpq | p (1 − p)² | 1 − f/2 | 1000 − 500f | 500f |
| PQqq | p² (1 − p) | (1 + f)/2 | 500 × (1 + f) | 500 × (1 − f) |
| PQpq | p (1 − p) | 0.5 | 500 | 500 |

μₖ˜Beta(μₖ|αₖ,βₖ), P is set as p allele frequency or 1/SNP number, and f is fetal concentration. This method can also be used to calculate fetal concentration.

22. After typing the free DNA in plasma of pregnant women and male genomic DNA data, each microhaplotype is compared to determine whether it matches the tested male. The supported sample microhaplotypes belong to one of the following situations:

| Genotype of biological | Genotype | Genotype of the |
|---|---|---|
| mother of child | of child | tested male |
| PP | PP | PP |
| PP | PQ | QQ |
| PP | PP | PQ |
| PP | PQ | QR |
| PP | PQ | PQ |
| PQ | QQ | QQ |
| PQ | QR | RR |
| PQ | QR | RS |
| PQ | PR | PR |
| PQ | QQ | QR |
| PQ | PQ | PP |
| PQ | PQ | QQ |
| PQ | PQ | PQ |
| PQ | PQ | PR |

(P, Q, R, S represent a certain genotype of microhaplotype; P, Q and R differ by only one SNP, S differs by multiple SNPs from P, Q, and R.)

Alternatively, gDNA sequencing analysis on maternal white blood cells is performed to improve the accuracy of maternal typing.

23. After typing the free DNA in plasma of pregnant women and male genomic DNA data, support situation, the parentage index is calculated by referring to the parentage

| Genotype of | | Genotype | Genotype | Calculation |
|---|---|---|---|---|
| biological | | of the | of the | formula of |
| mother of | Genotype | biological | tested | parentage |
| child | of child | father | male | index |
| PP | PP | P | PP | 1/p |
| PP | PQ | Q | QQ | 1/q |
| PP | PP | P | PQ | 1/(2p) |
| PP | PQ | Q | QR | 1/(2q) |
| PP | PQ | Q | PQ | 1/(2q) |
| PQ | QQ | Q | QQ | 1/q |
| PQ | QR | R | RR | 1/r |
| PQ | QR | R | RS | 1/(2r) |
| PQ | PR | R | PR | 1/(2r) |
| PQ | QQ | Q | QR | 1/(2q) |
| PQ | PQ | P or Q | PP | 1/(p + q) |
| PQ | PQ | P or Q | QQ | 1/(p + q) |
| PQ | PQ | P or Q | PQ | 1/(p + q) |
| PQ | PQ | P or Q | PR | 1/[2(p + q)] |

Note 1:
p, q, r represent the distribution frequencies of alleles P, Q, and R, respectively.

24. For unsupported loci, considering the fetal allele loss rate d, sequencing error rate e, and SNP mutation rate u, based on the actual situation, the allele loss rate d>e>u, and sequencing error e is generally 10⁻³ considering the characteristics of the proton platform, while u is generally 10⁻⁸. Due to the fact that it is common for two genotypes to differ by more than two SNPs in unsupported cases, and the probability of one microhaplotype simultaneously mutating two SNPs due to mutations in genetics is too low, it is believed value sequencing errors.

# it is divided into two categories, if a fetus is the same as a mother and the father is not detected, it is considered as lost; if the genotype of the fetus is different from that of the mother, it is considered sequencing errors.

| Genotype of | | | Genotype | Calculation |
|---|---|---|---|---|
| biological | | Genotype of | of the | formula of |
| mother of | Genotype | biological | tested | parentage |
| child | of child | father | male | index |
| PP | PP | P | QQ | d/p |
| PP | PQ | Q | PP | e/q |
| PP | PP | P | QS | d/(2p) |
| PQ | QQ/PP | P or Q | RR | d/(p + q) |
| PQ | QR/PR | R | PP/QQ | e/r |
| PQ | QQ/PP | P or Q | RS | d/2(p + q) |
| PQ | PQ | P or Q | SS | d^{(s-p)}/(p + q) |

Note 1:
p, q, r represent the distribution frequencies of alleles P, Q, and R, respectively;
Note 2:
P, Q and R differ by only one SNP, S differs by multiple SNPs from P, Q, and R;
Note 3:
(s − p) represents the number of SNPs that differ between S and P.

Alternative in step 24: for data with high sequencing quality, the number of unsupported sequences is added as an index of e into the formula for calculating sequencing errors.

25. The parentage index PI for each microhaplotype is calculated according to step 31 and step 32, and then the cumulative parentage index CPI for all sites as PI1×PI2×PI3× . . . ×PIn (1, 2, 3, and n represent the PI values of the 1st, 2nd, 3rd, and nth loci) is calculated. The calculation method of CPI also refers to the technical specification GB/T 37223-2018 for parentage identification.

26. Relationship determination: referring to the parentage identification technical specification GB/T 37223-2018, when the cumulative parentage index is lower than 0.0001, the hypothesis that the tested male (or the tested female) is not the biological father (or mother) of the child is supported. When the cumulative parentage index is greater than 10000, the hypothesis that the tested male (or the tested female) is the biological father (or mother) of the child is supported.

Alternative in determining the relationship: in step 29, a ratio F suspect of the numbers of supported and unsupported microhaplotypes for each pair of samples is obtained. The maternal and fetal typings obtained from free DNA of mother are compared with several other known unrelated males, and a ratio Fno relation of the number of supported and unsupported microhaplotypes with other males is counted. Chi square test is performed on F suspect and Fno relation, and if the p-value is lower than 0.01, it indicates a significant difference between the suspected paternal-fetal parentage relationship and the random stranger (male)-fetal parentage relationship; p-value<0.05 indicates significant difference; p-value>0.05 indicates that the difference is not significant. This chi square test can use other statistical methods, aiming to calculate the distribution relationship between F suspect and Fno relation. The advantage of this method lies in that when it is not possible to obtain gene frequency data for all microhaplotypes, it can reliably determine parentage relationship and is computationally simple.

Alternative: the above steps only use homozygous sites of the mother and fetus.

27. Determination of whether an egg is mistaken in assisted reproduction: infertility accounts for more than 10% of married couples in China, and assisted reproduction methods are needed to improve the success rate of fertility. Due to the fact that assisted reproduction process involves steps such as in vitro fertilization and embryo transfer that patients cannot supervise, concerns about whether sperm or egg has been mistaken have increased. If such samples can be identified, it can improve the experience of assisted reproduction process and also serve as a quality control step for assisted reproduction centers. Due to that assisted reproduction provides samples, the submitter needs to simultaneously identify the parentage relationship between the fetus and the pregnant woman, as well as the parentage relationship between the fetus and the expected sperm donor, it is not possible to directly use the above described steps, the same steps are used to obtain the microhaplotype typing of gDNA of the suspected father and free DNA of the pregnant woman (note that Y chromosome method or microhaplotype method is preferred for calculating fetal concentration, and SNP is not recommended. If the microhaplotype method is chosen, M_{two} should be used to calculate concentration simultaneously).

Alternative: comparing white blood cell DNA from pregnant women with free DNA, the excess free DNA is fetal DNA.

(1) The applicant needs to determine whether an egg belongs to a pregnant woman before comparing it with a male. If it belongs to the pregnant woman, the fetus should have a genotype from the mother, with the same method as described above; if it does not belong to the pregnant woman, the analysis method needs to be changed.

(2) Instructions for determining whether an egg belongs to a pregnant woman: in order to provide a more specific explanation of the analysis method, the applicant analyzes a specific site, a certain microhaplotype mh14CP003, genotype and population frequency are shown in the following figure. To simplify subsequent calculations, the applicant uses P. Q. R. Z to represent specific genotypes (other multi-genotypes can also be simplified in this way, Z is used to replace all genotypes after the top three genotype frequencies), and the genotype frequency is approximated to ¼.

| | | Population | Frequency |
|---|---|---|---|
| mh14CP003 | Genotype | frequency | substitution |
| P | GCG | 0.3 | 0.25 |
| Q | CTG | 0.24 | 0.25 |
| T | CCG | 0.005 | |
| R | CTA | 0.25 | 0.25 |
| S | GTG | 0.19 | |
| Z = T + S | CCG/GTG | 0.195 | 0.25 |

Then the phenotype of a person in this microhaplotype is shown in the following figure: in which homozygous has PP/QQ/RR/ZZ, four genotypes.

| Genotype | Frequency |
|---|---|
| PP | 0.0625 |
| PQ | 0.125 |
| PR | 0.125 |
| PZ | 0.125 |
| QQ | 0.0625 |
| QR | 0.125 |
| QZ | 0.125 |
| RR | 0.0625 |
| RZ | 0.125 |
| ZZ | 0.0625 |

If a fetus belongs to a mother and a fetus does not belong to a mother, in the case that the mother is homozygous PP, in the genotype and final peripheral blood of the child.

The data is shown in the following figure:

Fetus belongs to mother

| Genotype of | | |
|---|---|---|
| biological | | |
| mother of | Genotype | Occurrence |
| child | of child | probability |
| PP | PP | 0.015625 |
| PP | PQ | 0.015625 |
| PP | PR | 0.015625 |
| PP | PZ | 0.015625 |

| | |
|---|---|
| Fetus is homozygous | 0.015625 |
| Fetus is heterozygous | 0.046875 |
| Two genotypes of the fetus is | 0 |
| inconsistent with the mother | |
| Heterozygous and homozygous | 3 |
| proportion of fetuses | |

Fetus does not belong to mother

| Genotype of | | | |
|---|---|---|---|
| biological | | | |
| mother of | Genotype | | |
| child | of child | Phenotype | |
| PP | PP | PP\|PP | 0.00391 |
| PP | PQ | PP\|PQ | 0.00781 |
| PP | PR | PP\|PR | 0.00781 |
| PP | PZ | PP\|PZ | 0.00781 |
| PP | QQ | PP\|QQ | 0.00391 |
| PP | QR | PP\|QR | 0.00781 |
| PP | QZ | PP\|QZ | 0.00781 |
| PP | RR | PP\|RR | 0.00391 |
| PP | RZ | PP\|RZ | 0.00781 |
| PP | ZZ | PP\|ZZ | 0.00391 |

| | |
|---|---|
| Fetus is heterozygous | 0.05859 |
| Two genotypes of fetus is inconsistent with the mother | 0.02344 |
| Heterozygous and homozygous proportion of fetuses | 15 |

From the above two tables, it can be seen that, under the premise that a mother is homozygous, there is a significant difference in heterozygous sites of a fetus in terms of whether the fetus belongs to a pregnant woman, theoretically increasing the proportion by five times. In addition, when a fetus does not belong to a pregnant woman, two genotypes that the mother does not have will appear, which can also be used for identification. Alternatively, the difference can also be calculated when a mother is heterozygous.

(3) Determining whether an egg belongs to a pregnant woman, specific embodiment 1: after obtaining free DNA typing data of the peripheral blood of the pregnant women, the number of microhaploids of fetal heterozygous/maternal homozygous genotypes and the number of microhaploids of fetal homozygous/maternal homozygous genotypes are counted. The former is divided by the latter to obtain a ratio P_{hetero/homo}. Based on the known samples, it is determined in advance that the fetus belongs to the pregnant woman, that is, the normal pregnant sample set P_{hetero/homo}. Chi square validation is performed between unknown samples and the dataset. If it is lower than 0.001, it indicates that the fetus does not belong to the pregnant woman.

Preferably, fitting P_{hetero/homo} and fetal concentration, it can be found that the fetal concentration is positively correlated with the ratio, therefore, chi square validation after calibrating fetal concentration can calibrate the concentration effect.

Alternative: a threshold for P_{hetero/homo} is set via a sample set of normal pregnancies. If P_{hetero/homo} exceeds the set value, it is considered that the fetus does not belong to the pregnant woman herself.

(4) Determining whether an egg belongs to a pregnant woman, specific embodiment 2: in a singleton or monozygotic twin of normal pregnancy, since fetus has one allele from mother, there will not be two genotypes of fetus that are inconsistent with mother. As long as the result of the applicant's analysis shows that a fetus has two genotypes that are inconsistent with a mother, it is considered that the fetus does not belong to the pregnant woman herself. This method needs to be distinguished from dizygotic twins, as detailed below.

28. Determining whether twins are monozygotic: the genotype data in the preceding steps is also used, if a sample to be tested is twins with both double chorionic villi and double chorionic villi, it is impossible to distinguish between monozygotic twins and dizygotic twins. Given that there is a probability of the phenomenon of heteropaternal superfecundation at the same time in dizygotic twins, which can lead to errors in parentage identification results, and that step method 2 mentioned above is also affected by dizygotic twins, many parents are curious about whether the two children will grow the same when they find to be twins. In view of these reasons, the applicant has also compiled methods for determining whether twins are monozygotic. First, the genotype expression in free DNA of monozygotic twins and dizygotic twins is analyzed:

Homozygotic twins, due to the fact that they come from the same fertilized egg, they have the same genotype in peripheral blood as in singleton:

Monozygotic twin

| Genotype of | | |
|---|---|---|
| biological | | |
| mother of | Genotype | Occurrence |
| child | of child | probability |
| PP | PP | 0.015625 |
| PP | PQ | 0.015625 |
| PP | PR | 0.015625 |
| PP | PZ | 0.015625 |

| | |
|---|---|
| Fetus is homozygous | 0.015625 |
| Fetus is heterozygous | 0.046875 |
| Two genotypes of fetus is | 0 |
| inconsistent with the mother | |
| Heterozygous and homozygous | 3 |
| proportion of fetuses | |

### Dizygotic Twin:

Dizygotic twin

| Genotype of | | | | | |
|---|---|---|---|---|---|
| biological | | Prob- | | Prob- | |
| mother of | | ability | Genotype | ability | Final |
| child | Father | of father | of child | of twins | probability |
| PP | PP | 0.0625 | PP + PP | 1 | 0.00390625 |
| PP | PQ | 0.125 | PP + PP | 0.25 | 0.00195313 |
| PP | PQ | 0.125 | PP + PQ | 0.5 | 0.00390625 |
| PP | PQ | 0.125 | PQ + PQ | 0.25 | 0.00195313 |
| PP | PR | 0.125 | PP + PP | 0.25 | 0.00195313 |
| PP | PR | 0.125 | PP + PR | 0.5 | 0.00390625 |
| PP | PR | 0.125 | PR + PR | 0.25 | 0.00195313 |
| PP | PZ | 0.125 | PP + PP | 0.25 | 0.00195313 |
| PP | PZ | 0.125 | PP + PZ | 0.5 | 0.00390625 |
| PP | PZ | 0.125 | PZ + PZ | 0.25 | 0.00195313 |
| PP | QQ | 0.0625 | PQ + PQ | 1 | 0.00390625 |
| PP | QR | 0.125 | PQ + PQ | 0.25 | 0.00195313 |
| PP | QR | 0.125 | PQ + PR | 0.5 | 0.00390625 |
| PP | QR | 0.125 | PR + PR | 0.25 | 0.00195313 |
| PP | QZ | 0.125 | PQ + PQ | 0.25 | 0.00195313 |
| PP | QZ | 0.125 | PQ + PZ | 0.5 | 0.00390625 |
| PP | QZ | 0.125 | PZ + PZ | 0.25 | 0.00195313 |
| PP | RR | 0.0625 | PR + PR | 1 | 0.00390625 |
| PP | RZ | 0.125 | PR + PZ | 0.25 | 0.00195313 |
| PP | RZ | 0.125 | PR + PZ | 0.5 | 0.00390625 |
| PP | RZ | 0.125 | PZ + PZ | 0.25 | 0.00195313 |
| PP | ZZ | 0.0625 | PZ + PZ | 1 | 0.00390625 |

| | |
|---|---|
| Fetus is heterozygous | 0.00976563 |
| Fetus is homozygous | 0.05273438 |
| Two genotypes of the fetus are inconsistent with mother | 0.01171875 |
| Proportion of heterozygous and homozygous fetuses | 5.4 |

Through the above two comparisons, the applicant found that the best method to distinguish between monozygotic and dizygotic twins is to analyze a ratio of fetuses with two genotypes that are not consistent with mother's. In theory, monozygotic twins will not occur, in the case of occurrence that may be resulted from background errors, the ratio may be much lower than dizygotic twins, thus it can be used as the method to distinguish.

29. Determining whether a sperm is mistaken in assisted reproduction: after determining whether a fetus belongs to a pregnant woman in the preceding steps, the applicant identifies it according to different situations: if the fetus belongs to the pregnant woman, the applicant identifies parentage relationship according to steps 22-28; if the fetus does not belong to the pregnant woman, it is necessary to identify whether a sperm donor is the biological father of the fetus, or to find an egg donor, and adjust the method. For tested individuals whose fetuses do not belong to pregnant women, the biggest difference in the analysis is that if mother is PP and fetus is typed as PQ, the applicant cannot distinguish whether P comes from the mother, and therefore cannot determine based on whether the suspected father has Q. But for mother is PP and fetus is PP, in this case, it is speculated that the suspected father must have P in this microhaplotype; similarly, if mother is PP and fetus is PQ or QR, the biological father should also include at least one genotype of these two. In summary, after determining that a fetus does not belong to a pregnant woman, many coefficients of parentage contributed by genotype decrease, similar to the dyad in traditional parentage identification, referring to the way of parentage identification technical specification GB/T 37223-2018 to calculate the parentage index.

### EXAMPLES

1. Screening sites: this example is based on an ion proton platform, taking into account the characteristics of the proton platform, according to the selected microhaplotypes (see the preceding content for specific steps), the selected length is less than 160 bp, the internal sequences of microhaplotype does not have continuous repeat bases near SNPs, 295 loci in total.

2. Probe synthesis: the position information of each microhaplotype is organized into bed file format and submitted to NAANGDA (Nanjing) Biotechnology Co., Ltd. for design and synthesis by NAANGDA.

### Detailed Examples

The below samples are selected to perform experimental analysis:

| Sample | | | Week(s) of | |
|---|---|---|---|---|
| No. | Sample name | Sample type | pregnancy | Other |
| 1 | PT31360HA | Peripheral | / | Simulating |
| | | blood | | egg supply |
| 2 | PT31360HB | Peripheral | / | Simulating |
| | | blood | | sperm supply |
| 3 | PT31360W | Peripheral | / | Data from |
| | | blood | | non-pregnant |
| | | | | female |
| 4 | PT31693HB | Nail | / | / |
| 5 | PT31693W | White | 6 | / |
| | | blood cell | | |
| 6 | PT31693W | Peripheral | 6 | Dizygotic twin |
| | | blood | | |
| 7 | PT33097H | Peripheral | / | / |
| | | blood | | |
| 8 | PT33097W | Peripheral | 6 | / |
| | | blood | | |
| 9 | PT33202H | Hair | / | / |
| 10 | PT33202W | Peripheral | 10 | / |
| | | blood | | |

The same numerical value in the sample name represents a pair of samples, H: represents male, W: represents female, in which 31360W is non pregnant women who simulated surrogacy by adding data from simulated egg and sperm donors.

After conducting experiments, fastq file is obtained through high-throughput sequencing and subjected to basic analysis:

| Sample | | Raw | Average number |
|---|---|---|---|
| No. | Sample name | data | of layers |
| 1 | PT31360HA | 138760 | 57.7119 |
| 2 | PT31360HB | 525221 | 154.132 |
| 3 | PT31360W | 381923 | 86.3508 |
| 4 | PT31693HB | 222059 | 124.449 |
| 5 | PT31693W | 73210 | 48.5993 |
| 6 | PT31693W | 444165 | 88.6311 |
| 7 | PT33097H | 391893 | 134.386 |
| 8 | PT33097W | 510872 | 94.7697 |
| 9 | PT33202H | 360249 | 76.0393 |
| 10 | PT33202W | 588744 | 56.4045 |

### Preliminary Analysis of Fetal Concentration and Contamination of Male Samples

| Sample | | | Week(s) of | | Male |
|---|---|---|---|---|---|
| No. | Sample name | Sample type | pregnancy | FF-snp | contamination |
| 1 | PT31360HA | Peripheral blood | / | / | 7% |
| 2 | PT31360HB | Peripheral blood | / | / | 6% |
| 3 | PT31360W | Peripheral blood | / | 12% (mixed | |
| | | | | concentration) | |
| 4 | PT31693HB | Nail | / | / | 9% |
| 5 | PT31693W | White blood cell | / | / | / |
| 6 | PT31693W | Peripheral blood | 8 | 6% | / |
| 7 | PT33097H | Peripheral blood | / | / | 10% |
| 8 | PT33097W | Peripheral blood | 6 | 3% | / |
| 9 | PT33202H | Hair | / | / | 9% |
| 10 | PT33202W | Peripheral blood | 10 | 7% | / |

After typing males and females separately, they are arranged in the following format according to the same microhaplotype, and PI values are obtained by analyzing each site according to step 23. Each site can also be classified according to step 22. Word tables are not easy to type set, so screenshots are included. Some representative points have been selected here.

| | | PT33202W | | |
|---|---|---|---|---|
| | | Number of | Number of | Genotype |
| MH No. | Genotype | genotypes | microhaplotypes | frequency |
| Target-1 | ACGG | 2 | 37 | 0.05405405 |
| Target-1 | ATGC | 35 | 37 | 0.94594595 |
| Target-21 | GG | 55 | 55 | 1 |
| Target-270 | TGC | 44 | 44 | 1 |
| Target-17 | TGG | 36 | 37 | 0.97297297 |
| Target-17 | TGT | 1 | 37 | 0.02702703 |
| Target-268 | AC | 31 | 60 | 0.51666667 |
| Target-268 | GT | 29 | 60 | 0.48333333 |
| Target-373 | CCAGG | 3 | 28 | 0.10714286 |
| Target-373 | TCATA | 14 | 28 | 0.5 |
| Target-373 | TCGTG | 11 | 28 | 0.39285714 |
| Target-367 | CCCT | 10 | 16 | 0.625 |
| Target-367 | TCCC | 6 | 16 | 0.375 |
| Target-225 | ATAA | 17 | 38 | 0.44736842 |
| Target-225 | GCAG | 19 | 38 | 0.5 |

| | | PT33202H | | |
|---|---|---|---|---|
| | | Number of | Total | Genotype |
| MH No. | Genotype | genotypes | number | frequency |
| Target-1 | ACGG | 55 | 55 | 1 |
| Target-1 | | | | |
| Target-21 | GG | 82 | 82 | 1 |
| Target-270 | TGC | 38 | 83 | 0.45783133 |
| | TGT | 45 | 83 | 0.54216867 |
| Target-17 | TGG | 15 | 30 | 0.5 |
| Target-17 | TGT | 15 | 30 | 0.5 |
| Target-268 | GT | 73 | 73 | 1 |
| Target-268 | | | | |
| Target-373 | CCAGG | 44 | 44 | 1 |
| Target-373 | | | | |
| Target-373 | | | | |
| Target-367 | CCCT | 7 | 15 | 0.46666667 |
| Target-367 | TCCT | 8 | 15 | 0.53333333 |
| Target-225 | ACGG | 21 | 43 | 0.48837209 |
| Target-225 | GTGG | 21 | 43 | 0.48837209 |

| | | PT33202W | | | | | | |
|---|---|---|---|---|---|---|---|---|
| | | | Number of | | | PT33202H | | |
| | | Number of | microhap- | Genotype | | Number of | Total | Genotype |
| MH No. | Genotype | genotype | lotype | frequency | Genotype | genotype | number | frequency |
| Target-1 | ACGG | 2 | 37 | 0.05405405 | ACGG | 55 | 55 | 1 |
| Target-1 | ATGC | 35 | 37 | 0.94594595 | | | | |
| Target-21 | GG | 55 | 55 | 1 | GG | 82 | 82 | 1 |
| Target-270 | TGC | 44 | 44 | 1 | TGC | 38 | 83 | 0.45783133 |
| | | | | | TGT | 45 | 83 | 0.54216867 |
| Target-17 | TGG | 36 | 37 | 0.97297297 | TGG | 15 | 20 | 0.5 |
| Target-17 | TGT | 1 | 37 | 0.02702703 | TGT | 15 | 30 | 0.5 |
| Target-268 | AC | 31 | 60 | 0.51666667 | GT | 73 | 73 | 1 |
| Target-268 | GT | 29 | 60 | 0.48333333 | | | | |
| Target-373 | CCAGG | 3 | 28 | 0.10714286 | CCAGG | 44 | 44 | 1 |
| Target-373 | TCATA | 14 | 28 | 0.5 | | | | |
| Target-373 | TCGTG | 11 | 28 | 0.39285714 | | | | |
| Target-367 | CCCT | 10 | 16 | 0.625 | CCCT | 7 | 15 | 0.46666667 |
| Target-367 | TCCC | 6 | 16 | 0.375 | TCCT | 8 | 15 | 0.53333333 |
| Target-225 | ATAA | 17 | 38 | 0.44736842 | ACGG | 21 | 43 | 0.48837209 |
| Target-225 | GCAG | 19 | 38 | 0.5 | GTGG | 21 | 43 | 0.48837209 |

### Determining Parentage Relationship:

Method 1: CPI is calculated, which is determined by the genotype of sample itself and is also influenced by fetal concentration and number of layers during prenatal identification.

| Sample | CPI |
|---|---|
| PT33202W-PT33202H | 3.17*E77 |
| PT33202W-PT33097H | 5.3*E−55 |
| PT33097W-PT33097H | 2.3*E31 |
| PT31693W-PT31693H | 1.7*E74 |

### Method 2:

Due to a large number of MHs to be analyzed, we count the microhaplotypes that show inconsistent genotypes between fetus and mother and use these MHs to analyze whether they match suspected father(s), and calculate the proportion of matching and mismatching microhaplotypes to make a judgement, as described in step 26.

| Sample | Deny | Support | Support/Deny |
|---|---|---|---|
| PT33202W-PT33097H | 48 | 38 | 0.8 |
| PT33202W-PT33202H | 3 | 77 | 25.7 |
| PT33202W-PT31360HA | 47 | 41 | 0.9 |
| PT33202W-PT31360HB | 50 | 35 | 0.7 |
| PT33202W-PT31693H | 50 | 36 | 0.7 |

The above examples are preferred embodiments of the present invention, but the embodiments of the present invention are not limited by the above examples. Any other changes, modifications, substitutions, combinations, or simplifications made without departing from the spirit and principles of the present invention should be equivalent substitution methods and are included in the scope of protection of the present invention.

1. A non-invasive method for determining prenatal parentage relationship using microhaplotypes, the non-invasive method comprising:
1) screening sites, comprising the following steps of:
(1) pre-filtrating;
(2) identifying microhaplotypes;
(3) statistically analyzing genetic parameters of microhaplotype populations; and
(4) Hardy-Weinberg equilibrium testing,
2) constructing a target library by PCR amplification;
3) hybridization capturing and sequencing;
4) sample microhaplotyping; and
5) determining parentage relationship,
wherein in the step (2): after all pre-filtered SNPs are sorted by position, a first SNP is defined as “start SNP” and combined sequentially with subsequent SNPs, if a gap with the “start SNP” is within 350 bp, then they are combined to form a microhaplotype, with the “start SNP” and the number of SNPs being as unique marker;
if the gap between the SNPs and the “start SNP” exceeds 350 bp, marking the SNP next to the original “start SNP” as the “start SNP” and performing the above combination to identify each SNP in sequence;
for the microhaplotype of a certain “start SNP” that combines more than 2 SNPs, selecting the one with the most SNPs as the complete set and the others as subset, then removing the subset;
if the gap the between “start SNP” of the adjacent microhaplotypes is less than 350, that is, the microhaplotypes partially overlap, and are retained,
wherein in the step (4): for the selected microhaplotypes, Pearson chi square test is used to perform Hardy-Weinberg equilibrium test on a genotype distribution frequency of the microhaplotypes, and non-matching microhaplotype combinations are marked for selection based on subsequent applications, and
wherein the screening site further comprises an identification method using Cumulative parentage index or t-test, P-value to determine a parentage relationship.

2. The non-invasive method of claim 1, wherein in the step (1):
VCF files of a certain population or all races in a Thousand Genomes Project contain all mutation data, in allele frequency of the selected population, minor allele frequency is greater than 0.01; SNPs are located no autosomes and SNPs include tiny insertion and deletion.

3. The non-invasive method of claim 2, alternatively, wherein in the step (1), the insertion and deletion inDel are filtered out according to a sequencing platform.

4. (canceled)

5. The non-invasive method of claim 1, wherein in the step (2), the 350 bp is adjusted according to the selected reagents and experimental conditions, in prenatal parentage identification, due to short cf-DNA fragment, it is selected within 70-150 bp.

6. The non-invasive method of claim 1, wherein in the step (3):
for the above identified microhaplotypes, information of each SNP is found in the VCF files of (1), an effective number of alleles (Ae), informativeness (In), and allele frequency (P) of each microhaplotype are counted, if the Ae value of a certain genetic marker is n, it indicates that the genetic marker is equivalent to containing n alleles with equal frequencies, that is, a frequency of each allele is 1/n.

7. The non-invasive method of claim 6, wherein a calculation formula for Ae value is 1/Σpi², where pi represents the frequency of allele i on a certain locus, for the overlapping microhaplotypes in (2), the microhaplotype with higher Ae/Nₛₙₚ value are retained.

8. (canceled)

9. The non-invasive method of claim 1, wherein after this step is completed, there are several millions of the microhaplotypes, selecting is performed based on length, Ae, chromosome, and identification requirements.

10. The non-invasive method of claim 9, wherein in the step (4), two microhaplotypes with a gap of over 10 kb are selected.

11. (canceled)

12. The non-invasive method of claim 1, wherein the method comprises calibrating background noise of sequencing, wherein calculating background errors by the following: alignment results are call snp by genotyping software such as GATK to obtain VCF files, after removing SNPs contained in all the microhaplotypes, a number of bases in the remaining SNPs that are inconsistent with a reference genome is counted, and divided by a total number of bases aligned to the microhaplotypes in a sample, statistical and calibration methods for the background errors is achieved by methods of adding UMI.

13. The non-invasive method of claim 1, wherein the method comprises calculating fetal concentration: adding probes covering a Y chromosome and using a proportion of the Y chromosome to calculate the fetal concentration, denoted as FF_{y}; using a software FetalQuant to calculate the fetal concentration; using SeqFF algorithm to calculate the fetal concentration; using cfDNA fragment length information to calculate the fetal concentration; using a Nucleosome track method to calculate the fetal concentration; and using a methylation proportion to calculate the fetal concentration.

14. The non-invasive method of claim 1, wherein the method comprises analyzing sample contamination condition: evaluating whether a sample is contaminated by genotypes that are not at a reasonable frequency in male samples (under a premise of excluding experimental problems caused by), the genotypes is marked by the microhaplotypes or is marked by SNPs to analyze whether the sample is contaminated.

15. (canceled)

16. The non-invasive method for of claim 1, wherein the determining prenatal parentage relationship comprises analyzing conditions whether a fetus in singleton is mistaken sperm and/or egg.

17. The non-invasive method of claim 1, wherein the determining prenatal parentage relationship comprises analyzing conditions whether a fetus in twin is mistaken sperm and/or egg.

18. The non-invasive method of claim 1, wherein the determining prenatal parentage relationship comprises analyzing conditions whether a fetus in dizygotic twin is mistaken sperm and/or egg.

19. The non-invasive method of claim 1, wherein the determining prenatal parentage relationship comprises analyzing conditions whether a fetus in assisted reproduction is mistaken sperm and/or egg.

20. The non-invasive method of claim 1, wherein the determining prenatal parentage relationship comprises determining whether twins are monozygotic.

21. The non-invasive method of claim 1, wherein the sample microhaplotyping comprises microhaplotype typing of a male sample, a pregnant woman sample, or a fetus.
